(19) United States
(12) Patent Application Publication
(10) Pub. No.: US 2021/0330299 A1
(43) Pub. Date: Oct. 28, 2021

(54) Medical Swab

(71) Applicant: Rodon Limited Partnership, Hatfield, PA (US)

(72) Inventor: Joel I. Glickman, Jupiter, FL (US)

(21) Appl. No.: 17/224,896

(22) Filed: Apr. 7, 2021

Related U.S. Application Data
(60) Provisional application No. 63/014,255, filed on Apr. 23, 2020.

(51) Int. Cl. A61B 10/00

(52) U.S. Cl. CPC A61B 10/0051

(57) ABSTRACT

A medical swab comprising a unitary molding of plastic material, such as nylon or polyester, is configured to form an elongated stem and an enlarged tip portion at one end extremity of the stem. The enlarged tip portion is provided with a plurality of recesses in its outer surface for the reception and containment of biological material. The recesses are formed in two groups located on two opposing sides of said tip portion to accommodate production by molding.

### FIELD OF THE INVENTION

The invention relates to medical swabs of the type used for collecting biological specimens, for example from the nose and/or throat of a patient.

### BACKGROUND OF THE INVENTION

Medical swabs, which are in widespread use for the collection of specimens for clinical and diagnostic analysis, conventionally are of a two-part construction, comprised of a thin stem, which may be of wood or plastic, and a tip, usually formed of a hydrophilic fibrous material. Several manufacturing operations are required in the production of the conventional swabs. There is a need for an improved swab, which is more easily manufactured and is more suitable for the sterilized environment in which it is utilized.

### SUMMARY OF THE INVENTION

The medical swab of the invention is a single-piece molding of a suitable plastic material, such as polyester or nylon, which forms an elongated, small-diameter stem portion and an integral tip, which is enlarged in relation to the diameter of the stem portion. Pursuant to the invention, the enlarged tip is formed in the mold with a plurality of recesses of a size, shape and number to receive and retain a quantity of the biological material suitable for laboratory processing.

For a more complete understanding of the invention, reference can be made to the following detailed description of the new device, making reference to the accompanying drawings.

### BRIEF DESCRIPTION OF THE DRAWINGS

FIG. 1 is a side elevational view of a first embodiment of the molded, one-piece swab of the invention.

FIG. 2 is a top plan view of the embodiment of FIG. 1.

FIGS. 3 and 4 are enlarged views of the tip portion of the swab of FIG. 1, illustrating recesses therein for the reception and retention of biological material.

FIG. 5 is an orthographic view of the device of FIGS. 1-4.

FIGS. 6 and 7 are side elevational and top plan views respectively of a second embodiment of the invention, in which an outer end of the stem portion is of reduced diameter.

FIG. 8 is an enlarged view of the tip portion of the embodiment of FIGS. 6 and 7.

FIGS. 9 and 10 are enlarged top plan and side elevational views, respectively, of the tip portions of the device of FIGS. 6 and 7.

### DETAILED DESCRIPTION OF THE INVENTION

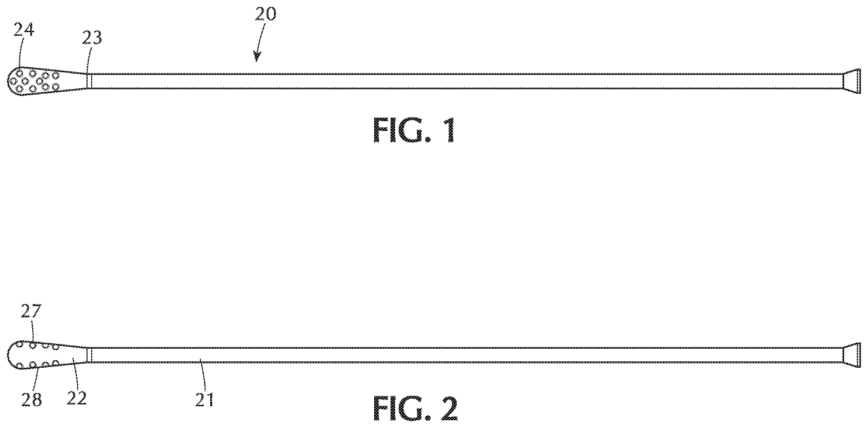

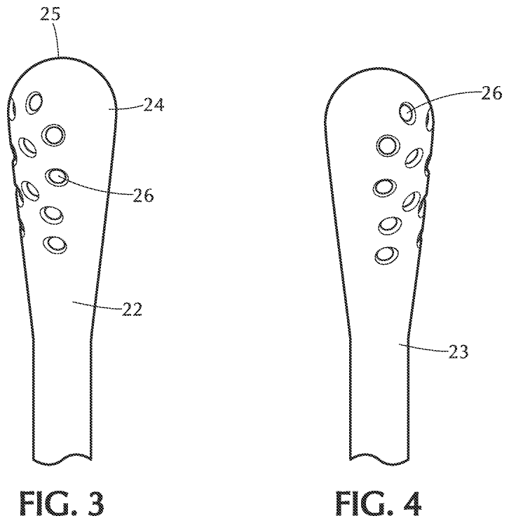

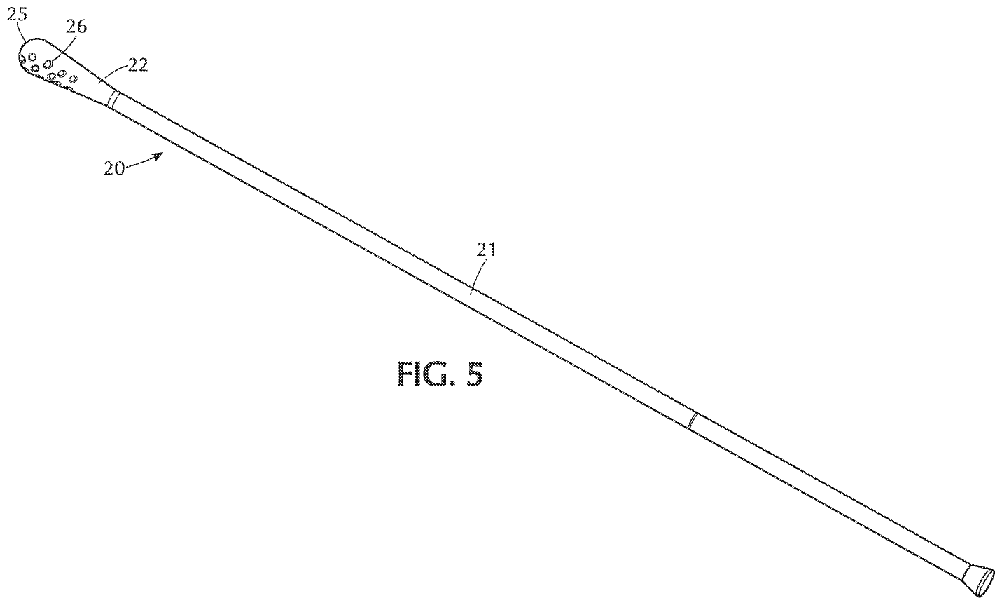

Referring initially to FIGS. 1-5, the reference numeral 20 designates generally a first embodiment of a medical swab according to the invention. The swab 20 is comprised of a stem portion 21 and an enlarged tip portion 22. Pursuant to the invention, the entire swab 20 is a one-piece molding of a suitable plastic material, preferably polyester or nylon. The stem portion 21 preferably may be about 5.25 inches in length, with a diameter of about 0.097 inch to provide a desired combination of strength and flexibility. The integral tip portion may have a length of about 0.625 inch, tapering from an initial diameter of about 0.056 inch at its inner end 23 to a maximum diameter of about 0.182 inch its outer end 24. The outer extremity 25 of the tip portion is smoothly semispherically rounded at a radius of 0.091 inch.

Pursuant to the invention, the tip portion is formed with a plurality of recesses 26, which may be approximately 0.032 inch in diameter and have a depth of, for example, 0.019 inch. In the illustrated form of the invention, the recesses 26 are arranged in two groups 27, 28, on opposite sides (which may be considered front and back) of the tip portion 22, with some space 29 between the two groups on the lateral sides of the tip portion, as can be seen in FIGS. 2-4 and 10. This enables mold projections (not shown), which form the recesses 26 during the molding process, to be withdrawn when the mold parts are separated to release the molded swabs. Desirably, the recesses 26 are of generally cylindrical form, defined by slightly tapered side walls, as distinguished from, for example, a shallow, dimpled configuration. This optimizes the ability of the recesses to receive and retain biological material for analysis, when the swab tips 22 are brought into contact with a surface to be tested, such a nasal passage, mouth, throat, etc.

In one preferred embodiment of the invention, the two recess groups 27, 28 each comprise a central set of three recesses, with a set of four recesses on either side of the central set. The end-most recess of the central set preferably is located on the rounded end 25 of the tip portion, for example about 0.040 inch axially from the end extremity of the tip 22. The end-most recesses of the side sets desirably are spaced 0.081 inch axially from the tip extremity, which positions those recesses slightly (i.e., 0.10 inch) forward of the maximum diameter of the tip. In an exemplary but non-limiting example, the three recesses of the central set may be spaced apart axially by 0.081 inch between the outermost and middle recesses, while the middle and innermost recesses are spaced apart axially by 0.096 inch. Axial spacing between the outermost and second recesses of the two side sets may be 0.091 inch, between the second and third recesses may be 0.086 inch, and between the third and fourth recesses 0.065 inch. The outermost and second recesses of the side sets may be spaced laterally by 0.048 inch from a theoretical axial center line defined by the central set of recesses, while the third and fourth recesses may be separated laterally by 0.035 inch from that center line. The various location measurements referred to herein for the various recesses are referenced from the centers of the respective recesses at the surface of the swab tip 22.

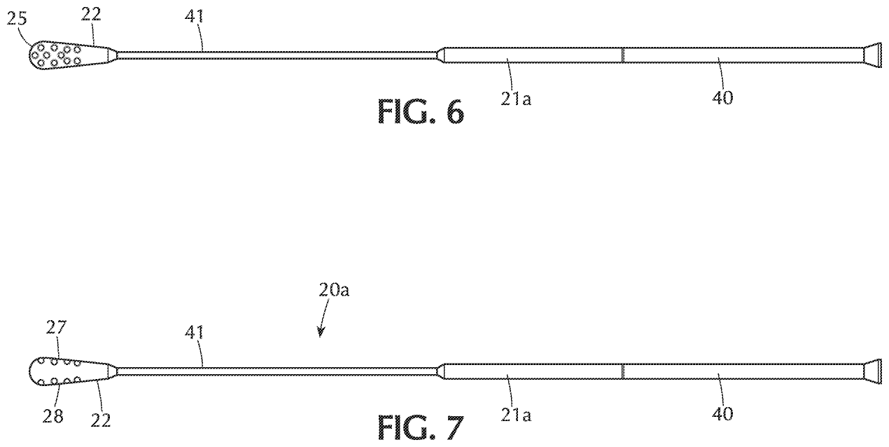

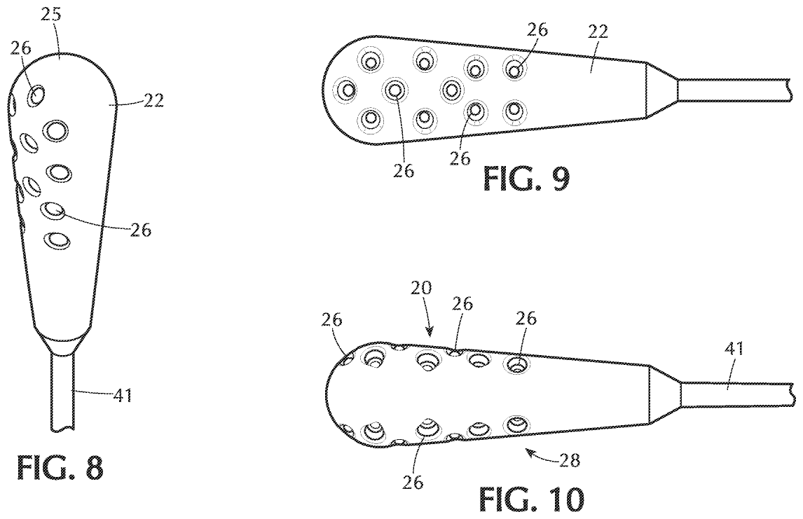

The embodiment of FIGS. 6-8 is identical to that of FIGS. 1-7, with exceptions to be noted, and corresponding reference numerals are applied to corresponding elements. In the embodiment of FIGS. 6-8, the stem portion 21a of the swab 20a is formed in two integral sections, 40 and 41. The inner or base end section 40 preferably may have a diameter of 0.097 inch, corresponding to that of the stem 21 of FIGS. 1-5. The outer section 41 of the stem 21a preferably is of a smaller diameter, for example 0.040 inch, in order to provide a greater degree of flexibility where necessary or desirable for patient comfort.

In the embodiment illustrated in FIGS. 6-8, the length of the base section 40 of the stem may be about 3.030 inches while the length of the small diameter outer section 41 may be about 2.15 inch.

In either of the disclosed embodiments, the one-piece swab of the invention incorporates a unique, integrally molded tip portion 22 which, on opposing sides thereof, incorporates a series of walled recesses for receiving and retaining biological material for examination. The new construction facilitates high volume, low cost manufacture of medical swabs.

1. A medical swab, comprised of a unitary molding of plastic material configured to form an elongated stem and an enlarged tip portion at one end extremity of the stem,
said enlarged tip portion having a having a plurality of recesses in an outer surface thereof for the reception and containment of biological material.

2. A medical swab according to claim 1, wherein
said plastic material is nylon or polyester.

3. A medical swab according to claim 1, wherein
said plurality of recesses on said enlarged tip portion are formed in two groups located on two opposing sides of said tip portion.

4. A medical swab according to claim 3, wherein
said plurality of recesses including a central set of recesses defining a theoretical center line and side sets of recesses on opposite sides of said center line.

5. A medical swab according to claim 4, wherein
said tip portion has an enlarged outer end portion of semi-spherical form and a body portion tapering toward said stem,
the respective side sets of recesses comprising recesses spaced at different distances from said centerline, and said side set recesses positioned closer to the enlarged outer end of said tip portion being spaced a greater distance from said center line than recesses of said side sets positioned closer to said stem.

6. A medical swab according to claim 5, wherein
each of said side sets of recesses comprises four recesses, with the axially outermost two of said side set recesses being spaced a greater distance from said center line than the axially innermost two of said side set recesses.

7. A medical swab according to claim 5, wherein
at least one recess of each of the central sets thereof being located on said semi-spherical outer portion of said tip portion.
